(19) United States
(12) Patent Application Publication
(10) Pub. No.: US 2016/0051787 A1
(43) Pub. Date: Feb. 25, 2016

(54) NASAL CANNULA

(71) Applicant: ATOM MEDICAL CORPORATION, Tokyo (JP)

(72) Inventors: Kazuo MATSUBARA, Tokyo (JP); Terumi MATSUBARA, Tokyo (JP); Kenji KOBAYASHI, Saitama-shi (JP); Shinichi KOBAYASHI, Saitama-shi (JP)

(21) Appl. No.: 14/711,281

(22) Filed: May 13, 2015

(30) Foreign Application Priority Data
Aug. 19, 2014 (JP) 2014-166541

(51) Int. Cl. A61M 16/06

(52) U.S. Cl. CPC A61M 16/0666

(57) ABSTRACT

A nasal cannula including: a cannula body having a tubular shape, which is provided with a pair of paths through which respiration gas flows, and a partition wall which partitions the paths and makes outer sides of bent parts of the paths respectively; a pair of introducing pipes which are formed integrally with the cannula body at both end parts of the cannula body so as to communicate with end parts of the paths respectively and to be connected to a pair of supply tubes supplying the respiration gas respectively; and a pair of nasal pipes which are formed integrally with the cannula body at a center part of the cannula body so as to communicate with other end parts of the paths respectively, and to be inserted into nares.

### RELATED APPLICATIONS

This application claims priority under 35 U.S.C. §119 to Japanese Patent Application No. 2014-166541 filed on Aug. 19, 2014, the entire content of which is hereby incorporated by reference.

### BACKGROUND OF THE INVENTION

1. Field of the Invention

The present invention relates to a nasal cannula for supplying respiration gas such as oxygen gas to a human body.

2. Description of the Related Art

Conventionally, artificial respirators sending respiration gas including specified quantity of oxygen to a respiratory tract of a patient, devices for oxygen inhalation therapy, and the like are known. The respiration gas sent from these devices is supplied to a human body through a nasal cannula.

As the nasal cannula, traditionally, in Japanese Unexamined Patent Application, First Publication No. 2003-38647 (hereinafter, “JP '647”) for example, a nasal cannula in which protruding parts (nasal pipes) which are inserted into nares are provided between a pair of end parts (introducing pipes) which are connected to tubes is disclosed. In this case, both the end parts communicate with each other in a left-and-right direction in the cannula body; and the two protruding parts in an adjacent state are connected to a middle of a path connecting both the end parts.

The respiration gas is supplied to the patient from the protruding parts of the nasal cannula by putting the cannula body on the patient, connecting both the end parts to a supply tube from the artificial respirator, and inserting the protruding parts into the nares of the patient.

### SUMMARY OF THE INVENTION

### Problems to be Solved by the Invention

When the nasal cannula of this type is used, ordinary, the respiration gas is supplied by a suitable quantity in accordance with expiration of the patient. However, in a case in which the respiration gas is forcibly supplied to the human body when the patient is in cardiopulmonary arrest, or for high-flow therapy (i.e., therapy supplying the respiration gas with a greater quantity than the expiration of the patient) which is recently featured, the respiration gas is supplied to the human body in a greater quantity than a usual. If the great quantity of the respiration gas is supplied to the human body using the conventional nasal cannula described in JP '648, because the quantity of the respiration gas is great, there is a problem in which large noise is generated when it flows in the nasal cannula.

Moreover, even though the respiration gas has adequate humidity, there is a risk that dew condensation could be generated in the nasal cannula because the quantity of flowing is great.

The present invention is achieved in consideration of the above circumstances, and has an object to provide a nasal cannula in which noise can be reduced even though a great quantity of respiration gas is supplied and dew condensation is unlikely to be generated.

### Means for Solving the Problem

A nasal cannula according to the present invention includes: a cannula body having a tubular shape, which is provided with a pair of paths through which respiration gas flows, and a partition wall which separates the paths and makes outer sides of bent parts in the paths respectively; a pair of introducing pipes which are formed integrally with the cannula body at both end parts of the cannula body so as to communicate with end parts of the paths respectively and to be connected to a pair of supply tubes supplying the respiration gas respectively; and a pair of nasal pipes which are formed integrally with the cannula body at a center part of the cannula body so as to communicate with other end parts of the paths respectively, and to be inserted into nares.

If the introducing pipes are communicated with each other in the cannula body by a single path as in the conventional nasal cannula, streams of the respiration gas supplied from the introducing pipes are struck against each other in the path of the cannula body; and causes a generation of noise. Since the two nasal pipes are connected to the part in which the introducing pipes are communicated, the respiration gas tends to stagnate between the nasal pipes, so that dew condensation is easy to be generated.

The nasal cannula of the present invention can prevent the generation of noise by separating the paths so as to communicate individually from the introducing pipes to the nasal pipes so that the streams of the respiration gas are not struck each other in the cannula body. Furthermore, the streams of the respiration gas supplied from the introducing pipes are guided direct to the nasal pipes respectively, so that the stagnation is not generated anywhere in the nasal cannula; as a result, the dew condensation can be prevented.

In the nasal cannula of the present invention, it is preferable that the partition wall include arc-shaped concave surfaces forming the outer sides of the bent parts in the paths.

Inner surfaces at the outer sides of the bent parts in the two paths are formed into the arc-shaped concave surfaces, so that the respiration gas can flow smoothly from the introducing pipes to the nasal pipes by being guided at the arc-shaped concave surfaces. Accordingly, the noise can be prevented more reliably from being generated. It is preferable that inner surfaces of the cannula body forming inner sides of the bent parts be formed into smooth arc-shaped convex surfaces, in addition to the surfaces of the outer sides of the bent parts formed concavely.

In the nasal cannula of the present invention, it is preferable that a plurality of ribs extending along longitudinal directions of the nasal pipes be formed with circumferential intervals on inner peripheral surfaces at end parts of the nasal pipes.

Since the nasal pipes are inserted into the nares of the human body. they are formed thin and flexible so as not to harm the human body. The respiration gas is blown out from opening ends of the nasal pipes at high-flow quantity. Therefore, the respiration gas tends to vibrate when being blown out from the nasal pipes, so that there is a case in which sound is generated by the vibration.

In the nasal cannula according to the present invention, the ribs are formed on the nasal pipes, thereby improving rigidity and preventing the vibration though they are thin and flexible as a whole. Moreover, by flowing between parallel ribs to each other, the respiration gas is rectified, thereby preventing turbulent flow from being generated. As a result, the generation of the noise can be reduced. Furthermore, by forming the ribs, pipe shapes of the nasal pipes can be maintained since inner surface thereof are not in contact even when the thin nasal pipes are squashed.

### Effects of the Invention

According to the nasal cannula of the present invention, by individually separating the paths extending from the introducing pipes to the nasal pipes so that the streams of the respiration gas are not struck each other in the cannula body, the respiration gas can flow smoothly from the introducing pipes to the nasal pipes. Accordingly, the generation of the noise can be prevented even though the respiration gas is supplied at high-flow quantity. Moreover, since it is a structure in which the respiration gas does not easily stagnate in the cannula body, the dew condensation can be also prevented from being generated.

### BRIEF DESCRIPTION OF THE DRAWINGS

FIG. 1 is an upper view showing an embodiment of a nasal cannula according to the present invention.

FIG. 2 is a frontal view of FIG. 1.

FIG. 3 is a left-side view of FIG. 1.

FIG. 4 is a cross-sectional view taken along the line A-A in FIG. 1.

FIG. 5 is a cross-sectional view taken along the line B-B in FIG. 1.

FIG. 6 is an enlarged cross-sectional view taken along the line C-C in FIG. 1.

FIG. 7 is an enlarged perspective view showing a vicinity of an opening-end part of a nasal pipe.

FIG. 8 is a frontal view showing a state in which the nasal cannula is put on a human body.

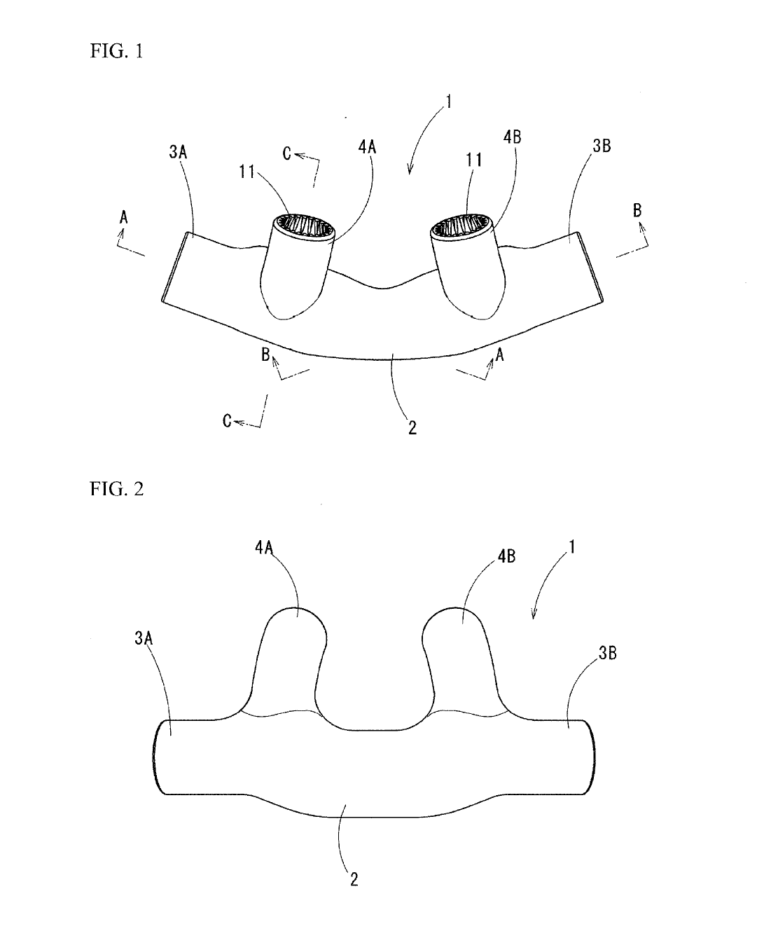

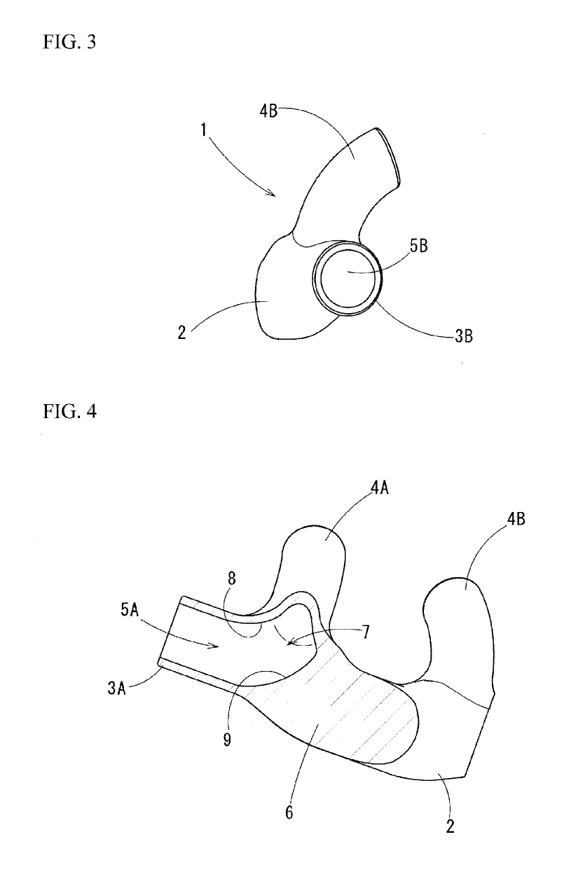

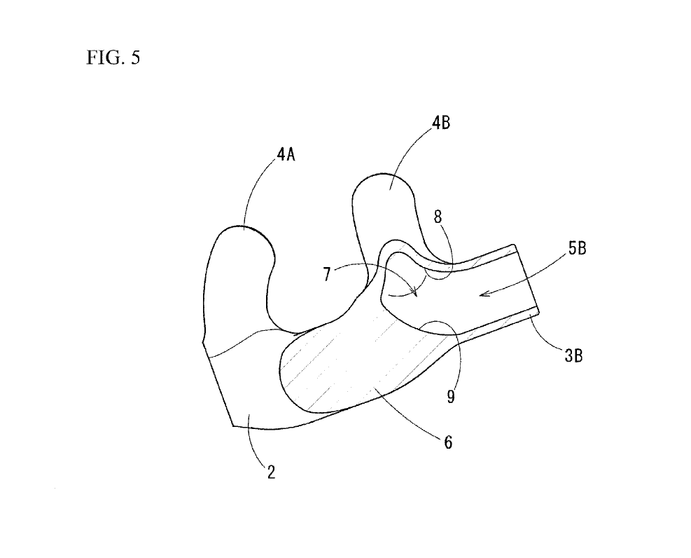

### DETAILED DESCRIPTION OF THE PREFERRED EMBODIMENTS

Below, an embodiment of a nasal cannula according to the present invention will be described with reference to the drawings.

The drawings show a nasal cannula 1 of an embodiment. A perpendicular direction and a crosswise direction are defined by a state shown in a frontal view of FIG. 2.

The nasal cannula 1 is provided with: a cannula body 2 extending along the crosswise direction; and introducing pipes 3A and 3B formed at both end parts of the cannula body 2 and connected to below-mentioned supplying tubes 15 for respiration gas respectively. Between the introducing pipes 3A and 3B, a pair of nasal pipes 4A and 4B which are inserted into nares are provided upward in FIG. 2. The nasal cannula 1 is entirely formed from soft synthetic resin such as polystyrene elastomer, silicone rubber, urethane, and the like.

The introducing pipes 3A and 3B and the nasal pipes 4A and 4B are communicated with each other in pairs respectively. One path 5A connects the left introducing pipe 3A and the left nasal pipe 4A; and the other path 5B connects the right introducing pipe 3B and the right nasal pipe 4B. In this case, the introducing pipes 3A and 3B are formed into substantially a straight pipe shape: and the nasal pipes 4A and 4B are formed to be bent in an arc-shape of the same direction to each other and to be slightly inclined with respect to the perpendicular direction so that opening-ends approach each other.

The cannula body 2 is formed to be bent at a center part of the crosswise direction so as to be parallel to an outside of a face when it is put on a human body. The center part is solid except parts of the introducing pipes 3A and 3B at both the left and right ends. The solid part is a partition wall 6 which separates the two paths 5A and 5B which connect the introducing pipes 3A and 3B and the nasal pipes 4A and 4B. By providing the partition wall 6 at the center part of the cannula body 2, the paths 5A and 5B are formed to be bent in the cannula body 2 along the crosswise direction from the introducing pipes 3A and 3B at both the left and right end parts, bent upward in FIG. 2, and connected to the nasal pipes 4A and 4B.

Inner surfaces of the cannula body 2 facing inner sides and outer sides of bent parts 7 in the paths 5A and 5B are formed into arc surface so as to form the smooth bent parts 7. That is to say: the inner surfaces of the cannula body 2 forming the inner sides of the bent parts 7 are formed into arc-shaped convex surfaces 8; and surfaces of the partition wall 6 forming the outsides of the bent parts 7 are formed into arc-shaped concave surfaces 9, so that the paths 5A and 5B are smoothly guided from the introducing pipes 3A and 3B to the nasal pipes 4A and 4B. Accordingly, in the paths 5A and 5B, by forming the inner sides of the bent parts 7 into the arc-shaped convex surfaces 8 and the outer sides of the bent parts 7 into the arc-shaped concave surfaces 9, the arc-shaped bent parts 7 are formed as a whole so as to smoothly connect between the introducing pipes 3A and 3B and the nasal pipes 4A and 4B.

Although radius of curvature and the like of the arc-shaped convex surfaces 8 and the arc-shaped concave surfaces 9 are not limited; for example, the radius of curvature of the arc-shaped convex surfaces 8 is set to 5 mm and the radius of curvature of the arc-shaped concave surfaces 9 is set to 8.5 mm with respect to an inner diameter 5.5 mm of the paths 5A and 5B.

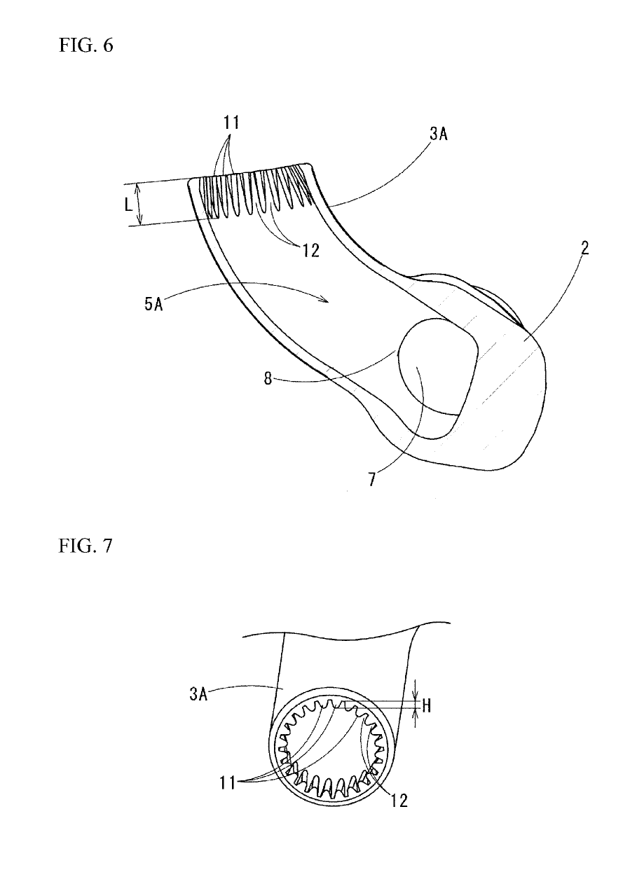

On inner peripheral surfaces at distal ends of the nasal pipes 4A and 4B, a plurality of ribs 11 are formed along a longitudinal direction of the nasal pipes 4A and 4B (i.e., flow directions of the respiration gas) as shown in FIG. 6 and FIG. 7 with enlarging. These ribs 11 are formed to have semicircular cross-section at top parts with a maximum height at the opening ends of the nasal pipes 4A and 4B, and the height is gradually reduced toward inside the nasal pipes 4A and 4B as illustrated. These ribs 11 are formed along a circumferential direction, so that grooves 12 are formed between the ribs 11.

Although size of the ribs 11 is not specifically limited; for example, with respect to the nasal pipes 4A and 4B having an inner diameter of 5.0 mm, the ribs 11 are formed to have a height H of 0.4 mm and a length L of 2.0 mm with intervals of 15° in the circumferential direction.

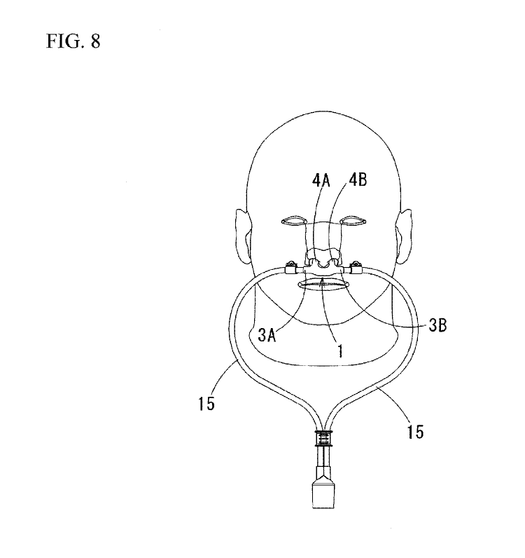

The nasal cannula 1 as configured above is, as shown in FIG. 8, put on the human body in a state in which: the introducing pipes 3A and 3B are connected to the supplying tubes 15 for the respiration gas respectively; the supplying tubes 15 are connected to an artificial respirator (not illustrated); and the nasal pipes 4A and 4B are inserted into the nares. The respiration gas is supplied from the artificial respirator to the left and right introducing pipes 3A and 3B through the supplying tubes 15, and supplied to the human body from the nasal pipes 4A and 4B through the inner paths 5A and 5B.

In supplying the respiration gas, even in a case in which the respiration gas is forcibly supplied or a case in which the respiration gas is supplied at a high-flow quantity for what is called a high-flow therapy, since the two paths 5A and 5B from the introducing pipes 3A and 3B to the nasal pipes 4A and 4B are separated by the partition wall 6, streams of the respiration gas of the high-flow quantity which are introduced from the left and right direction are not struck each other in the cannula body 2. Although the paths 5A and 5B are bent in the cannula body 2, the respiration gas can be smoothly guided since the smooth bent parts 7 are formed, by forming the inner surface of the cannula body 2 at the inner sides and the outer sides of the bent parts 7 into the arc-shaped convex surfaces 8 and the arc-shaped concave surfaces 9. Moreover, since the plurality of ribs 11 are formed on the inner peripheral surfaces at the opening-end parts of the nasal pipes 4A and 4B, vibration is prevented from being generated, and the respiration gas is blown out with being rectified by passing through the grooves 12 between the ribs 11. By these mutual effects, the nasal cannula 1 scarcely generates noise and can be used for the patient at ease even though the respiration gas is supplied at the high-flow quantity.

As described above, if the left and right paths are communicated with each other in the cannula body, the dew condensation is generated, so that there is a possibility that the dew condensation water flows down into the nares of the lying patient. However, in the nasal cannula 1 of the present embodiment, the dew condensation is not generated since the respiration gas does not stagnate in the cannula body 2 by a structure in which the two paths 5A and 5B are separated by the partition wall 6. In this case, since the paths 5A and 5B are smoothly bent between the introducing pipes 3A and 3B and the nasal pipes 4A and 4B by the arc-shaped bent parts 7, the respiration gas flows smoothly, so that the inside stagnation can be more reliably prevented.

Since the ribs 11 are formed at the opening-end parts of the nasal pipes 4A and 4B, the inner peripheral surfaces of the nasal pipes 4A and 4B are not contact nor clogged even when the nasal pipes 4A and 4B are squashed, so that the pipe-shapes of the nasal pipes 4A and 4B are not spoiled, and soundness thereof can be maintained.

Although both the surfaces of the inner sides and the outer sides of the bent parts 7 in the paths 5A and 5B are formed into the arc-shape surfaces respectively, it is important that at least the surfaces of the outer sides are formed into the arc-shaped concave surfaces 9. Since the respiration gas flows along the surfaces of the arc-shaped concave surfaces 9 when passing through the bent parts 7, it flows smoothly and the generation of the noise can be prevented. Needless to say, also in the arc-shaped convex surfaces 8 at the surfaces of the inner sides of the bent parts 7, there is an effect for the respiration gas to flow without straying off from the wall surface, so that it is effective for preventing the generation of the noise.

An embodiment of the present invention is described above. The present invention is not limited to the above-described embodiment, and various modifications and revision may be made based on the scope of the claimed invention.

What is claimed is:

1. A nasal cannula comprising:
a cannula body having a tubular shape, which is provided with a pair of paths through which respiration gas flows, and a partition wall which separates the paths and makes outer sides of bent parts in the paths respectively;
a pair of introducing pipes which are formed integrally with the cannula body at both end parts of the cannula body so as to communicate with end parts of the paths respectively and to be connected to a pair of supply tubes supplying the respiration gas respectively; and
a pair of nasal pipes which are formed integrally with the cannula body at a center part of the cannula body so as to communicate with other end parts of the paths respectively, and to be inserted into nares.

2. The nasal cannula according to claim 1, wherein the partition wall comprises arc-shaped concave surfaces forming the outer sides of the bent parts in the paths.

3. The nasal cannula according to claim 1, a plurality of ribs extending along longitudinal directions of the nasal pipes are formed with circumferential intervals on inner peripheral surfaces at end parts of the nasal pipes.

4. The nasal cannula according to claim 2, wherein the cannula body comprises arc-shaped convex surfaces forming inner sides of the bent parts of the paths.
